SEQUENCE LISTING (47 sequences)

SEQ ID NO: 1 - protein, 13 aa, Artificial
  VH CDR3
LTHYARYYRYFDV

SEQ ID NO: 2 - protein, 17 aa, Artificial
  VH CDR3
GKGNTHKPYGYVRYFDV

SEQ ID NO: 3 - protein, 15 aa, Artificial
  VH CDR3
LLSRGYNGYYHKFDV

SEQ ID NO: 4 - protein, 8 aa, Artificial
  VL-CDR3
QQVYNPPV

SEQ ID NO: 5 - protein, 8 aa, Artificial
  VL-CDR3
FQLYSDPF

SEQ ID NO: 6 - protein, 8 aa, Artificial
  VL-CDR3
QQLSSFPP

SEQ ID NO: 7 - protein, 122 aa, Artificial
  VH
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARLTHYARYYRYFDVWGQGTLVTVSS

SEQ ID NO: 8 - protein, 126 aa, Artificial
  VH
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGNTHKPYGYVRYFDVWGQGTLVTVSS

SEQ ID NO: 9 - protein, 124 aa, Artificial
  VH
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARLLSRGYNGYYHKFDVWGQGTLVTVSS

SEQ ID NO: 10 - protein, 110 aa, Artificial
  VL
DIVLTQSPATLSLSPGERATLSCRASQSVSSSYLAWYQQKPGQAPRLLIYGASSRATGVPARFSGSGSGTDFTLTISSLEPEDFAVYYCQQVYNPPVTFGQGTKVEIKRT

SEQ ID NO: 11 - protein, 110 aa, Artificial
  VL
DIVLTQSPATLSLSPGERATLSCRASQSVSSSYLAWYQQKPGQAPRLLIYGASSRATGVPARFSGSGSGTDFTLTISSLEPEDFATYYCFQLYSDPFTFGQGTKVEIKRT

SEQ ID NO: 12 - protein, 110 aa, Artificial
  VL
DIVLTQSPATLSLSPGERATLSCRASQSVSSSYLAWYQQKPGQAPRLLIYGASSRATGVPARFSGSGSGTDFTLTISSLEPEDFATYYCQQLSSFPPTFGQGTKVEIKRT

SEQ ID NO: 13 - DNA, 19 bp, Artificial
  primer #37
caggagagtgtcacagagc

SEQ ID NO: 14 - DNA, 18 bp, Artificial
  primer #38
ctctttgtgacgggcgag

SEQ ID NO: 15 - DNA, 19 bp, Artificial
  primer #62
ctccctcagcagcgtggtg

SEQ ID NO: 16 - DNA, 18 bp, Artificial
  primer #63
gctcacgtccaccaccac

SEQ ID NO: 17 - DNA, 19 bp, Artificial
  primer #64
gcattatgcacctccacgc

SEQ ID NO: 18 - DNA, 21 bp, Artificial
  primer #65
gcggctttgtcttggcattat

SEQ ID NO: 19 - DNA, 18 bp, Artificial
  primer #66
gcgtcctcaccgtcctgc

SEQ ID NO: 20 - DNA, 23 bp, Artificial
  primer #67
caagtgcaaggtctccaacaaag

SEQ ID NO: 21 - DNA, 20 bp, Artificial
  primer #68
ccattgctctcccactccac

SEQ ID NO: 22 - DNA, 20 bp, Artificial
  primer #131
ctgttgtgtgcctgctgaat

SEQ ID NO: 23 - DNA, 20 bp, Artificial
  primer #132
gacttcgcaggcgtagactt

SEQ ID NO: 24 - DNA, 20 bp, Artificial
  primer #133
tcacagagcaggacagcaag

SEQ ID NO: 25 - DNA, 18 bp, Artificial
  primer #134
tgctttgctcagcgtcag

SEQ ID NO: 26 - DNA, 20 bp, Artificial
  primer #139
ctggaactgcctctgttgtg

SEQ ID NO: 27 - DNA, 20 bp, Artificial
  primer #145
tgacgctgagcaaagcagac

SEQ ID NO: 28 - DNA, 18 bp, Artificial
  primer #146
caggccctgatgggtgac

SEQ ID NO: 29 - DNA, 28 bp, Artificial
  FAM-primer #147-TAMRA
acgagaaacacaaagtctacgcctgcga

SEQ ID NO: 30 - DNA, 24 bp, Artificial
  primer #148
caaaggcacagtcaaggctgagaa

SEQ ID NO: 31 - DNA, 24 bp, Artificial
  primer #149
tggtgaagacgccagtagattcca

SEQ ID NO: 32 - DNA, 24 bp, Artificial
  FAM-primer #165-BHQ1
cctccaatcgggtaactcccagga

SEQ ID NO: 33 - DNA, 24 bp, Artificial
  FAM-primer #166-BHQ1
agcacctacagcctcagcagcacc

SEQ ID NO: 34 - DNA, 24 bp, Artificial
  IRD700-primer #167-BHQ3
atcacaagcccagcaacaccaagg

SEQ ID NO: 35 - DNA, 23 bp, Artificial
  IRD700-primer #168-BHQ3
atctccaaagccaaagggcagcc

SEQ ID NO: 36 - DNA, 21 bp, Artificial
  primer #169
attgtggaaggactcatgacc

SEQ ID NO: 37 - DNA, 20 bp, Artificial
  primer #170
gatgcagggatgatgttctg

SEQ ID NO: 38 - DNA, 20 bp, Artificial
  Yakima Yellow-priomer #171-BHQ1
cctccggaaagctgtggcgt

SEQ ID NO: 39 - DNA, 24 bp, Artificial
  Yakima Yellow-primer #172-BHQ1
ccatcactgccacccagaagactg

SEQ ID NO: 40 - DNA, 23 bp, Artificial
  Cy5-primer #173-BHQ3
atctccaaagccaaagggcagcc

SEQ ID NO: 41 - DNA, 23 bp, Artificial
  Yakima Yellow-primer #174-BHQ1
agatcccgccaacatcaaatggg

SEQ ID NO: 42 - DNA, 24 bp, Artificial
  Yakima Yellow-primer #175-BHQ1
aacatcaaatggggtgatgctggc

SEQ ID NO: 43 - DNA, 24 bp, Artificial
  primer #176-BHQ1
aacatcaaatggggtgatgctggc

SEQ ID NO: 44 - DNA, 323 bp, Homo sapiens
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 45 - DNA, 294 bp, Homo sapiens
cctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttg

SEQ ID NO: 46 - DNA, 330 bp, Homo sapiens
cacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaag

SEQ ID NO: 47 - DNA, 323 bp, Homo sapiens
ggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggcaaatga

(19) United States
(12) Patent Application Publication
(10) Pub. No.: US 2011/0229903 A1
(43) Pub. Date: Sep. 22, 2011

(54) DETERMINATION OF IMMUNOGLOBULIN ENCODING NUCLEIC ACID

(76) Inventors: Ulrich Goepfert, PENZBERG (DE); Marlene Jentzsch, Pinnow (DE); Andrea Osterlehner, Penzberg (DE)

(21) Appl. No.: 13/124,400

(22) PCT Filed: Oct. 21, 2009
(86) PCT No.: PCT/EP09/07521

§ 371 Date: Jun. 1, 2011

(30) Foreign Application Priority Data
Oct. 23, 2008 (EP) 08018698.4

(51) Int. Cl. C12Q 1/68; C07H 21/04; C12P 21/00

(52) U.S. Cl. 435/6.12; 536/24.33; 435/71.1

(57) ABSTRACT

It is reported herein a method for the determination of the amount of immunoglobulin-encoding mRNA comprising: a) providing a sample, b) performing a polymerase chain reaction for amplifying the light chain with the primers of SEQ ID NO: and 24 and the probe of SEQ ID NO: 33, and/or c) performing a polymerase chain reaction for amplifying the heavy chain with the primers of SEQ ID NO: 19 and 21 and the probe of SEQ ID NO: 40, and d) quantitating with an efficiency of 2.0. The primers with SEQ ID NOs 23 and 24 bind at positions CL 247-266 and CL166-185, respectively, and the probe with SEQ ID NO: 33 binds at 189-212 in human IgG koppa chain. The primer with SEQ ID NO: 19 binds at CH region 2 position 220-237 and the primer with SEQ ID NO: 21 binds at CH region 3 position 114-133. Finally the probe with SEQ ID NO: 40 binds from position 315 in CH2 to position 7 in CH3.

The current invention is directed to a method for the determination of immunoglobulin encoding nucleic acid, i.e. RNA and DNA, and primers for PCR determination of immunoglobulin encoding nucleic acid.

### BACKGROUND OF THE INVENTION

In current biotechnological processes genetically engineered microorganism are employed in order to provide therapeutical polypeptides in high yield. The Chinese hamster ovary (CHO) cell line is widely used for the production of recombinant polypeptides, especially therapeutic immunoglobulins. This cell line is capable of providing secondary modifications and most importantly the CHO cell line is capable of secreting the recombinantly produced polypeptide to the culture medium facilitating down stream process operations (Jiang, Z., et al., Biotechnol. Prog. 22 (2006) 313-138; Yee, J. C., et al., Biotechnol. Bioeng. 102 (2009) 246-263). In order to increase the productivity of recombinant cell lines parameters like the parental cell line, the cultivation medium, or the cultivation conditions have to be optimized (Yee, J. C., et al., Biotechnol. Bioeng. 102 (2009) 246-263).

Based on the analysis of position, structure and copy number of integrated heterologous nucleic acids in the genome of the recombinant cell line indicators for the decision about the recombinant cell lines properties shall be established (Wurm, F. M., Ann. N. Y. Acad. Sci. 782 (1996) 70-78). The nucleic acid encoding the heterologous polypeptide is integrated into the genome of the recombinant cell line as deoxyribonucleic acid (DNA), which is transcribed into ribonucleic acid (RNA) during the transcription process. The RNA is in turn the template for protein biosynthesis in the translation process. Due to the importance of the RNA for gene expression, analysis of this nucleic acid gains importance (Seth, G., et al., Biotechnol Bioeng. 97 (2007) 933-951).

In WO 2008/094871 a method for the selection of high producing cell lines is reported. A study of monoclonal antibody-producing CHO cell lines is reported by Chusainow, J., et al. (Biotechnol. Bioeng. 102 (2009) 1182-1196). Barnes, L. M., et al. (Biotechnol. Bioeng. 85 (2004) 115-121) report molecular definition of predictive indicators of stable protein expression in recombinant NSO myeloma cells.

### SUMMARY OF THE INVENTION

One aspect of the current invention is a method for the determination of the amount of mRNA encoding an immunoglobulin light chain and/or an immunoglobulin heavy chain of the IgG1 or IgG4 subclass with a polymerase chain reaction and absolute quantitation, by
a) performing a polymerase chain reaction for the immunoglobulin light chain with the primers of SEQ ID NO: 23 and 24 and the probe of SEQ ID NO: 33 with the dye FAM in a TaqMan hydrolysis probe format, and/or
b) performing a polymerase chain reaction for the immunoglobulin heavy chain with the primers of SEQ ID NO: 19 and 21 and the probe of SEQ ID NO: 40 with the dye Cy5 in a TaqMan hydrolysis probe format, and
c) performing absolute quantitation with an efficiency of 2.0.

Further aspects of the current invention are a first kit comprising the nucleic acids of SEQ ID NO: 23, SEQ ID NO: 24 and SEQ ID NO: 33 and a second kit comprising the nucleic acids of SEQ ID NO: 19, SEQ ID NO: 21 and SEQ ID NO: 40. Another aspect is the use of the nucleic acids of SEQ ID NO: 23, 24, and 33 or of SEQ ID NO: 19, 21, and 40 in a polymerase chain reaction.

Another aspect of the current invention is a method for determining the productivity of a cell expressing a heterologous polypeptide comprising the following steps in the following order:
determining the amount of mRNA encoding said heterologous polypeptide in a cell of known productivity,
determining the amount of mRNA encoding said heterologous polypeptide in a cell of unknown productivity,
calculating the ratio of the determined amount of mRNA encoding said heterologous polypeptide in said cell of unknown productivity to said cell of known productivity,
multiplying the productivity of said cell of known productivity with said calculated ratio and thereby determining the productivity of a cell expressing a heterologous polypeptide.

In one embodiment said heterologous polypeptide is an immunoglobulin, or immunoglobulin fragment, or immunoglobulin conjugate. In still a further embodiment said determining of said amount of mRNA is via a polymerase chain reaction (PCR). In one embodiment the determining the amount of mRNA is by a polymerase chain reaction with the primers of SEQ ID NO: 23 and 24 and the probe of SEQ ID NO: 33 with the dye FAM in a TaqMan hydrolysis probe format and/or by a polymerase chain reaction with the primers of SEQ ID NO: 19 and 21 and the probe of SEQ ID NO: 40 with the dye Cy5 in a TaqMan hydrolysis probe format. In a further embodiment said amount of mRNA encoding said heterologous immunoglobulin is the average of the amount of mRNA encoding the light chain of said heterologous immunoglobulin and the amount of mRNA encoding the heavy chain of said heterologous immunoglobulin. In another embodiment said productivity is the specific production rate in pg/cell/day. In another embodiment said polymerase chain reaction is a multiplex polymerase chain reaction.

### DETAILED DESCRIPTION OF THE INVENTION

In the current invention it has been found that the copy number of an immunoglobulin encoding nucleic acid (DNA) and the amount of transcript generated there from (RNA) can be used to determine the productivity of a recombinant CHO cell line expressing a heterologous immunoglobulin. Also has been found that the amount of mRNA encoding a heterologous polypeptide is a measure for the specific productivity of such a cell.

The invention comprises a method for determining the productivity of a cell expressing an immunoglobulin comprising
a) performing a polymerase chain reaction with the primers of SEQ ID NO: 23 and 24 and the probe of SEQ ID NO: 33, and/or performing a polymerase chain reaction with the primers of SEQ ID NO: 19 and 21 and the probe of SEQ ID NO: 40 and thereby determining the amount of mRNA encoding the immunoglobulin in a cell of known productivity,
b) performing a polymerase chain reaction with the primers of SEQ ID NO: 23 and 24 and the probe of SEQ ID NO: 33, and/or performing a polymerase chain reaction with the primers of SEQ ID NO: 19 and 21 and the probe of SEQ ID NO: 40 and thereby determining the amount of mRNA encoding the immunoglobulin in a cell of unknown productivity,
c) calculating the ratio of the determined amount of mRNA encoding the immunoglobulin of the cell of unknown productivity to the cell of known productivity,
d) multiplying the productivity of the cell of known productivity with the calculated ratio and thereby determining the productivity of a cell expressing an immunoglobulin.

Methods and techniques known to a person skilled in the art, which are useful for carrying out the current invention, are described e.g. in Ausubel, F. M., ed., Current Protocols in Molecular Biology, Volumes I to III (1997), Wiley and Sons; Sambrook, et al., Molecular Cloning: A Laboratory Manual, Second Edition, Cold Spring Harbor Laboratory Press, Cold Spring Harbor, N.Y. (1989).

The term “amino acid” as used within this application denotes the group of carboxy α-amino acids, which directly or in form of a precursor can be encoded by a nucleic acid. The individual amino acids are encoded by nucleic acids consisting of three nucleotides, so called codons or base-triplets. Each amino acid is encoded by at least one codon. The encoding of the same amino acid by different codons is known as “degeneration of the genetic code”. The term “amino acid” as used within this application denotes the naturally occurring carboxy α-amino acids and is comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “nucleic acid” or a “nucleic acid sequence”, which terms are used interchangeably within this application, refers to a polymeric molecule consisting of individual nucleotides (also called bases) A, C, G and T (or U in RNA), for example to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a cell.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “immunoglobulin” encompasses the various forms of immunoglobulin structures including complete immunoglobulins and immunoglobulin conjugates. The immunoglobulin employed in the current invention is in one embodiment a human antibody, or a humanized antibody, or a chimeric antibody, or a T cell antigen depleted antibody (see e.g. WO 98/33523, WO 98/52976, and WO 00/34317). Genetic engineering of immunoglobulins is e.g. described in Morrison, S. L., et al., Proc. Natl. Acad. Sci. USA 81 (1984) 6851-6855; U.S. Pat. No. 5,202,238 and U.S. Pat. No. 5,204,244; Riechmann, L., et al., Nature 332 (1988) 323-327; Neuberger, M. S., et al., Nature 314 (1985) 268-270; Lonberg, N., Nat. Biotechnol. 23 (2005) 1117-1125. Immunoglobulins may exist in a variety of formats, including, for example, Fv, Fab, and F(ab)₂ as well as single chains (scFv) or diabodies (e.g. Huston, J. S., et al., Proc. Natl. Acad. Sci. USA 85 (1988) 5879-5883; Bird, R. E., et al., Science 242 (1988) 423-426; in general, Hood et al., Immunology, Benjamin N.Y., 2nd edition (1984); and Hunkapiller, T. and Hood, L., Nature 323 (1986) 15-16).

The term “complete immunoglobulin” denotes an immunoglobulin which comprises two so called light chains and two so called heavy chains. Each of the heavy and light chains of a complete immunoglobulin contains a variable domain (variable region) (generally the amino terminal portion of the polypeptide chain) comprising binding regions that are able to interact with an antigen. Each of the heavy and light chains of a complete immunoglobulin comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The variable domain of an immunoglobulin's light and heavy chain in turn comprises different segments, i.e. four framework regions (FR) and three hypervariable regions (CDR).

The term “immunoglobulin conjugate” denotes a polypeptide comprising at least one domain of an immunoglobulin heavy or light chain conjugated via a peptide bond to a further polypeptide. The further polypeptide is a non-immunoglobulin peptide, such as a hormone, or growth receptor, or antifusogenic peptide, or complement factor, or the like. Exemplary immunoglobulin conjugates are reported in WO 2007/045463.

The term “heterologous immunoglobulin” denotes an immunoglobulin which is not naturally produced by a mammalian cell or the host cell. The immunoglobulin produced according to a method of the invention is produced by recombinant means. Such methods are widely known in the state of the art and comprise protein expression in eukaryotic cells with subsequent recovery and isolation of the heterologous immunoglobulin, and usually purification to a pharmaceutically acceptable purity. For the production, i.e. expression, of an immunoglobulin a nucleic acid encoding the light chain and a nucleic acid encoding the heavy chain are inserted each into an expression cassette by standard methods. Nucleic acids encoding immunoglobulin light and heavy chains are readily isolated and sequenced using conventional procedures. Hybridoma cells can serve as a source of such nucleic acids. The expression cassettes may be inserted into a(n) expression plasmid(s), which is (are) then transfected into host cells, which do not otherwise produce immunoglobulins. Expression is performed in appropriate prokaryotic or eukaryotic host cells and the immunoglobulin is recovered from the cells after lysis or from the culture supernatant.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e. at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

“Heterologous DNA” or “heterologous polypeptide” refers to a DNA molecule or a polypeptide, or a population of DNA molecules or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter. A peptide or polypeptide encoded by a non-host DNA molecule is a “heterologous” peptide or polypeptide.

The term “cell” or “host cell” refers to a cell into which a nucleic acid, e.g. encoding a heterologous polypeptide, can be or is transfected. The term “cell” includes both prokaryotic cells, which are used for propagation of plasmids, and eukaryotic cells, which are used for the expression of a nucleic acid and production of the encoded polypeptide. In one embodiment, the eukaryotic cells are mammalian cells. In another embodiment the mammalian cell is a CHO cell, preferably a CHO K1 cell (ATCC CCL-61 or DSM ACC 110), or a CHO DG44 cell (also known as CHO-DHFR[-], DSM ACC 126), or a CHO XL99 cell, a CHO-T cell (see e.g. Morgan, D., et al., Biochemistry 26 (1987) 2959-2963), or a CHO-S cell, or a Super-CHO cell (Pak, S. C. O., et al., Cytotechnology. 22 (1996) 139-146). If these cells are not adapted to growth in serum-free medium or in suspension an adaptation prior to the use in the current method is to be performed. As used herein, the expression “cell” includes the subject cell and its progeny. Thus, the words “transformant” and “transformed cell” include the primary subject cell and cultures derived there from without regard for the number of transfers or subcultivations. It is also understood that all progeny may not be precisely identical in DNA content, due to deliberate or inadvertent mutations. Variant progeny that have the same function or biological activity as screened for in the originally transformed cell are included.

The term “expression” as used herein refers to transcription and translation processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR or by Northern hybridization (see Sambrook, et al., 1989, supra). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook, et al., 1989, supra).

Expression of a gene is performed either as transient or as permanent expression. The polypeptide of interest is in general a secreted polypeptide and therefore contains an N-terminal extension (also known as the signal sequence) which is necessary for the transport/secretion of the polypeptide through the cell wall into the extracellular medium. In general, the signal sequence can be derived from any gene encoding a secreted polypeptide. If a heterologous signal sequence is used, it preferably is one that is recognized and processed (i.e. cleaved by a signal peptidase) by the host cell. For secretion in yeast for example the native signal sequence of a heterologous gene to be expressed may be substituted by a homologous yeast signal sequence derived from a secreted gene, such as the yeast invertase signal sequence, alpha-factor leader (including Saccharomyces, Kluyveromyces, Pichia, and Hansenula α-factor leaders, the second described in U.S. Pat. No. 5,010,182), acid phosphatase signal sequence, or the C. albicans glucoamylase signal sequence (see EP 0 362 179). In mammalian cell expression the native signal sequence of the protein of interest is satisfactory, although other mammalian signal sequences may be suitable, such as signal sequences from secreted polypeptides of the same or related species, e.g. for immunoglobulins from human or murine origin, as well as viral secretory signal sequences, for example, the herpes simplex glycoprotein D signal sequence. The DNA fragment encoding for such a presegment is ligated in frame, i.e. operably linked, to the DNA fragment encoding a polypeptide of interest.

The transfection of e.g. a CHO cell according to the method according to the invention is performed as sequential steps of transfection and selection. CHO cells suitable in the method according to the invention are e.g. a CHO K1 cell, or a CHO DG44 cell, or a CHO XL99 cell, or a CHO DXB11 cell, or a CHO DP12 cell, or a super-CHO cell. Within the scope of the present invention, transfected cells may be obtained with substantially any kind of transfection method known in the art. For example, the nucleic acid may be introduced into the cells by means of electroporation or microinjection. Alternatively, lipofection reagents such as FuGENE 6 (Roche Diagnostics GmbH, Germany), X-tremeGENE (Roche Diagnostics GmbH, Germany), LipofectAmine (Invitrogen Corp., USA), and nucleotransfection (AMAXA Corp.) may be used. Still alternatively, the nucleic acid may be introduced into the cell by appropriate viral vector systems based on retroviruses, lentiviruses, adenoviruses, or adeno-associated viruses (Singer, O., Proc. Natl. Acad. Sci. USA 101 (2004) 5313-5314).

Usually, gene expression profiling on the DNA or RNA level is monitored on routine basis by a multi-step procedure. First, the respective cellular sample is removed from the culture vessel. In case of adherent cells harvesting may be supported by trypsination (treatment with a Trypsin-EDTA solution) in order to detach the adherent cells from the solid support. Secondly, the collected cells are pelleted and subjected to cell lysis. As a third step it is usually required to at least partially purify the total RNA, mRNA or DNA that is present in the sample (e.g. see EP 0 389 063). Afterwards, if required, a first strand cDNA synthesis step is performed with an RNA dependent DNA polymerase such as AMV or MoMULV Reverse Transcriptase (Roche Applied Science, Germany).

Subsequently, the amount DNA or of generated cDNA is quantified either by means of quantitative PCR (Sanger, G. and Goldstein, C., Biochemica 3 (2001) 15-17) or alternatively by means of amplification and subsequent hybridization onto a DNA microarray (Kawasaki, E. S., Ann. N.Y. Acad. Sci. 1020 (2004) 92-100). In case of polymerase chain reaction (PCR), a one step RT-PCR may be performed, characterized in that the first strand cDNA synthesis and subsequent amplification are catalyzed by the same Polymerase such as T.th Polymerase (Roche Applied Science Cat. No. 11 480 014, Germany).

In one embodiment the gene expression analysis is based on real time PCR. Such a monitoring in real time is characterized in that the progress of amplification of the nucleic acid in the PCR reaction is monitored and quantitated in real time. Different detection formats are known in the art. The below mentioned detection formats have been proven to be useful for PCR and thus provide an easy and straight forward possibility for gene expression analysis:

a) TaqMan Hydrolysis probe format:

A single-stranded hybridization probe is labeled with two components. When the first component is excited with light of a suitable wavelength, the absorbed energy is transferred to the second component, the so-called quencher, according to the principle of fluorescence resonance energy transfer. During the annealing step of the PCR reaction, the hybridization probe binds to the target DNA and is degraded by the 5′-3′ exonuclease activity of the Taq Polymerase during the subsequent elongation phase. As a result the excited fluorescent component and the quencher are spatially separated from one another and thus a fluorescence emission of the first component can be measured. TaqMan probe assays are reported in detail in U.S. Pat. No. 5,210,015, U.S. Pat. No. 5,538,848, and U.S. Pat. No. 5,487,972. TaqMan hybridization probes and reagent mixtures are reported in U.S. Pat. No. 5,804,375.

b) Molecular Beacons:

These hybridization probes are labeled with a fluorescent component and a quencher, the labels preferably being located at both ends of the probe. As a result of the secondary structure of the probe, both components are in spatial vicinity in solution. After hybridization to the target nucleic acids both components are separated from one another such that after excitation with light of a suitable wavelength the fluorescence emission of the first component can be measured (U.S. Pat. No. 5,118,801).

c) FRET hybridization probes:

The FRET hybridization probe test format is especially useful for all kinds of homogenous hybridization assays (Matthews, J. A. and Kricka, L. J., Anal. Biochem. 169 (1988) 1-25). It is characterized by two single-stranded hybridization probes which are used simultaneously and which are complementary to adjacent sites of the same strand of the amplified target nucleic acid. Both probes are labeled with different fluorescent components. When excited with light of a suitable wavelength, a first component transfers the absorbed energy to the second component according to the principle of fluorescence resonance energy transfer (FRET) such that a fluorescence emission of the second component can be measured when both hybridization probes bind to adjacent positions of the target molecule to be detected. Alternatively to monitoring the increase in fluorescence of the FRET acceptor component, it is also possible to monitor fluorescence decrease of the FRET donor component as a quantitative measurement of a hybridization event.

In particular, the FRET hybridization probe format may be used in real time PCR, in order to detect the amplified target DNA. Among all detection formats known in the art of real time PCR, the FRET-Hybridization Probe format has been proven to be highly sensitive, exact and reliable (see WO 97/46707; WO 97/46712; WO 97/46714). As an alternative to two FRET hybridization probes, it is also possible to use a fluorescent-labeled primer and only one labeled oligonucleotide probe (Bernard, P. S., et al., Anal. Biochem. 255 (1998) 101-107). In this regard, it may be chosen arbitrarily, whether the primer is labeled with the FRET donor or the FRET acceptor compound.

d) SYBR® Green format:

It is also within the scope of the invention that if real time PCR is performed in the presence of an additive that in case the amplification product is detected using a double stranded nucleic acid binding moiety. For example, the respective amplification product can also be detected according to the invention by a fluorescent DNA binding dye, which emits a corresponding fluorescence signal upon interaction with the double-stranded nucleic acid after excitation with light of a suitable wavelength. The dyes SYBR® Green I and SYBR® Gold (Molecular Probes, USA) have proven to be particularly suitable for this application. Intercalating dyes can alternatively be used. However, for this format, in order to discriminate the different amplification products, it is necessary to perform a respective melting curve analysis (U.S. Pat. No. 6,174,670).

e) Multiplex format:

The simultaneous determination of different nucleic acids in one reaction vessel is termed multiplex real time PCR. Generally for the determination of each nucleic acid a fluorescence dye not interfering or having only a small overlap with the other employed dyes is required.

The PCR primers used in the current invention and which are also aspects of the invention were designed with the software eprimer3 according to the following parameters:
specific binding to the sequence to be amplified,
no or unlikely primer dimer formation,
length between 18 and 25 nucleotides,
G/C content of approximately 50%,
melting temperature of approximately 60° C.,
amplicon of 500 basepairs or less, in one embodiment between 100 and 250 base pairs,
preferably the primers should bind to neighboring exons and the PCR product should span at least one intron to enable discrimination between amplification of genomic DNA and cDNA.

The nucleic acids complementary to the designed primers are located within the constant regions of immunoglobulins heavy and light chains identical in IgG1 and IgG4 type immunoglobulins.

The probes used in the method are also an aspect of the current invention and were designed with the software eprimer3 according to the following parameters:
melting temperature of approximately 70° C.,
no G at the 5′ end,
no or unlikely dimer formation with primers or other probes,
preferably the probes intended to be used for RT PCR should bind to two different adjacent exons to enable discrimination between amplification of genomic DNA and cDNA.

In one embodiment the nucleic acids complementary to the designed probes are located within the constant regions of immunoglobulins heavy and light chains identical in IgG1 and IgG4 type immunoglobulins. The probes were labeled in order to allow for a multiplex RT-PCR reaction as follows:
light chain: fluorescent dye FAM, excitation at 465 nm, detection at 510 nm,
reference gene: Yakima Yellow dye, excitation at 533 nm, detection at 580 nm,
heavy chain: fluorescent dye IRD 700 or Cy5, excitation at 618 nm, detection at 660 nm.

The primers and probes listed in Table 1 were designed and are each individually and as combination an aspect of the current invention.

TABLE 1
Primers and probes.

| | | x- | Tm | SEQ ID |
|---|---|---|---|---|
| # | Primer/probe sequence (5′-3′) | mer | [° C.] | NO: |
| 37 | CAGGAGAGTGTCACAGAGC | 19 | 58.8 | 13 |
| 38 | CTCTTTGTGACGGGCGAG | 18 | 58.2 | 14 |
| 62 | CTCCCTCAGCAGCGTGGTG | 19 | 63.1 | 15 |
| 63 | GCTCACGTCCACCACCAC | 18 | 60.5 | 16 |
| 64 | GCATTATGCACCTCCACGC | 19 | 58.8 | 17 |
| 65 | GCGGCTTTGTCTTGGCATTAT | 21 | 57.9 | 18 |
| 66 | GCGTCCTCACCGTCCTGC | 18 | 62.8 | 19 |
| 67 | CAAGTGCAAGGTCTCCAACAAAG | 23 | 60.6 | 20 |
| 68 | CCATTGCTCTCCCACTCCAC | 21 | 61.4 | 21 |
| 131 | CTGTTGTGTGCCTGCTGAAT | 20 | 58 | 22 |
| 132 | GACTTCGCAGGCGTAGACTT | 20 | 60 | 23 |
| 133 | TCACAGAGCAGGACAGCAAG | 20 | 60 | 24 |
| 134 | TGCTTTGCTCAGCGTCAG | 18 | 56 | 25 |
| 139 | CTGGAACTGCCTCTGTTGTG | 20 | 60 | 26 |
| 145 | TGACGCTGAGCAAAGCAGAC | 20 | 60 | 27 |
| 146 | CAGGCCCTGATGGGTGAC | 18 | 61 | 28 |
| 147 | (FAM)-ACGAGAAACACAAAGTCTACGCCTGCGA-(TAMRA) | 28 | 70 | 29 |
| 148 | CAAAGGCACAGTCAAGGCTGAGAA | 24 | 65 | 30 |
| 149 | TGGTGAAGACGCCAGTAGATTCCA | 24 | 65 | 31 |
| 165 | (FAM)-CCTCCAATCGGGTAACTCCCAGGA-(BHQ1) | 24 | 69 | 32 |
| 166 | (FAM)-AGCACCTACAGCCTCAGCAGCACC-(BHQ1) | 24 | 70 | 33 |
| 167 | (IRD700)-ATCACAAGCCCAGCAACACCAAGG-(BHQ3) | 24 | 67 | 34 |
| 168 | (IRD700)-ATCTCCAAAGCCAAAGGGCAGCC-(BHQ3) | 23 | 66 | 35 |
| 169 | ATTGTGGAAGGACTCATGACC | 21 | 59 | 36 |
| 170 | GATGCAGGGATGATGTTCTG | 20 | 58 | 37 |
| 171 | (Yakima Yellow)-CCTCCGGAAAGCTGTGGCGT-(BHQ1) | 20 | 65 | 38 |
| 172 | (Yakima Yellow)-CCATCACTGCCACCCAGAAGACTG-(BHQ1) | 24 | 69 | 39 |
| 173 | (Cy5)-ATCTCCAAAGCCAAAGGGCAGCC-(BHQ3) | 23 | 66 | 40 |
| 174 | (Yakima Yellow)-AGATCCCGCCAACATCAAATGGG-(BHQ1) | 23 | 65 | 41 |
| 175 | (Yakima Yellow)-AACATCAAATGGGGTGATGCTGGC-(BHQ1) | 24 | 65 | 42 |
| 176 | (HEX)-AACATCAAATGGGGTGATGCTGGC-(BHQ1) | 24 | 65 | 43 |

The location of the primers and probes in the immunoglobulin constant region is shown in FIGS. 1 to 4.

In the following the current invention is exemplified based on three cell lines producing an immunoglobulin specifically binding to the amyloid β-A4 peptide (anti-Aβ antibody), whereby the first cell line is transfected once, the second cell line is transfected two times, and the third cell line is transfected three times with a plasmid containing a nucleic acid encoding the immunoglobulin.

The gene expression of the heavy and light immunoglobulin chain was determined with RT-PCR by quantitation of the heavy and light chain mRNAs in the constant region encoding part using the dye SYBR® Green I and TaqMan probes. The determination is in one embodiment performed with total cell RNA.

The determination of the mRNA amount of the light antibody chain of the three cell lines was independently performed five times each with three different mRNA amounts of 250 ng, 50 ng, and 10 ng and the dye SYBR® Green I. The result of one representative experiment obtained with the primer combination #131 and #132 is listed based on the mRNA amount of the single transfected cell line 8C8, which was set to 100% relative amount in Table 2. It can be seen, that the twice transfected cell line 4F5 has approximately 40% more mRNA encoding immunoglobulin light chain than the single transfected cell line, and that the thrice transfected cell line 20F2 has approximately 70% more mRNA encoding the immunoglobulin light chain.

TABLE 2
Exemplary results with primer combination #131 and #132.

| amount of mRNA | cell line 4F5 | |
|---|---|---|
| in the sample | % relative to cell line | cell line 20F2 |
| [ng] | 8C8 ± σ | % relative to cell line 8C8 ± σ |
| 250 | 140.44 ± 8.36 | 178.18 ± 5.34 |
| 50 | 143.06 ± 17.51 | 160.03 ± 20.18 |
| 10 | 145.40 ± 25.79 | 166.63 ± 34.76 |
| relative average | 142.97 ± 2.48 | 168.28 ± 9.19 |
| value | | |

The above performed determination method is specific as only a single product is obtained as confirmed by agarose gel electrophoresis and shown in FIG. 5.

For the determination of the mRNA amount of the light antibody chain of the three cell lines with TaqMan hydrolysis probes at first the combination of primers and probe useful in this aspect of the invention had to be determined. The combinations listed in the Table 3 were tested.

TABLE 3
Tested TaqMan format nucleic acid.

primer #

| forward | reverse | TaqMan probe # |
|---|---|---|
| 139 | 134 | 165, 166 |
| 139 | 132 | 165, 166 |
| 139 | 146 | 147, 165, 166 |
| 139 | 38 | 147, 165, 166 |
| 145 | 146 | 147 |
| 145 | 38 | 165 |
| 131 | 134 | 165, 166 |
| 131 | 132 | 165, 166 |
| 131 | 146 | 147, 165, 166 |
| 131 | 38 | 147, 165, 166 |
| 37 | 134 | 166 |
| 37 | 132 | 166 |
| 37 | 146 | 147, 166 |
| 37 | 38 | 166 |
| 133 | 134 | 166 |
| 133 | 132 | 166 |
| 133 | 146 | 147, 166 |
| 133 | 38 | 166 |

The PCR products obtained with the different primer-probe-combinations as listed above show (e.g. FIG. 6) that the combinations primers #133 and #132 with probe #166 as well as the combination primers #133 and #38 with probe #166 resulted in PCR products with a high specific product yield and low by-product formation. Thus, the primer-probe-combinations #133, #132, and #166 as well as the primer-probe-combination #133, #38, #166 itself are specific aspects of the current invention as well as the use of these primer-probe-combinations. In one embodiment is the primer-probe-combination #133, #132, and #166. This combination is preferred as it shows a better PCR efficiency, i.e. a steeper increase of the amplification curve as denoted in FIG. 7.

The determination of the mRNA amount of the light antibody chain of the three cell lines was independently performed four times each with three different mRNA amounts of 250 ng, 50 ng, and 10 ng. The result of one representative experiment obtained with the primer combination #133/#132 and the probe #166 is listed based on the mRNA amount of the single transfected cell line, which was set to 100% relative amount in Table 4. It can be seen, that the cell line 4F5 has approximately 77% more mRNA encoding immunoglobulin light chain than the single transfected cell line, and that the cell line 20F2 has approximately 114% more mRNA encoding the immunoglobulin light chain.

TABLE 4
Exemplary results with primer-probe-combination #133/#132/#166.

| amount of mRNA | cell line 4F5 | |
|---|---|---|
| in the sample | % relative to cell line | cell line 20F2 |
| [ng] | 8C8 ± σ | % relative to cell line 8C8 ± σ |
| 250 | 171.51 ± 16.83 | 211.4 ± 15.40 |
| 50 | 183.08 ± 9.22 | 213.61 ± 5.32 |
| 10 | 177.15 ± 7.14 | 219.62 ± 8.85 |
| relative average | 177.25 ± 5.78 | 214.88 ± 4.25 |
| value | | |

The above performed determination method is specific as only a single product is obtained as confirmed by agarose gel electrophoresis.

For the determination of the mRNA amount of the heavy antibody chain the primers #62 and #65 and the dye SYBR® Green I were used. These primers bind to two different exons (CH1- and CH2 region, respectively), which are separated by one intron, the hinge-exon and a second intron.

The determination of the mRNA amount of the heavy antibody chain of the three cell lines was independently performed three times each with three different mRNA amounts of 250 ng, 50 ng, and 10 ng. The above performed determination method is specific as only a single product is obtained as confirmed by agarose gel electrophoresis and shown in FIG. 8.

The result of one representative experiment obtained with the primer combination #62/#65 is listed based on the mRNA amount of the single transfected cell line, which was set to 100% relative amount in Table 5. It can be seen, that the cell line 4F5 has approximately 60% more mRNA encoding immunoglobulin light chain than the single transfected cell line, and that the cell line 20F2 has approximately 140% more mRNA encoding the immunoglobulin light chain.

TABLE 5
Exemplary results with primer combination #62/#65.

| amount of mRNA | cell line 4F5 | |
|---|---|---|
| in the sample | % relative to cell line | cell line 20F2 |
| [ng] | 8C8 ± σ | % relative to cell line 8C8 ± σ |
| 250 | 129.83 ± 17.01 | 174.11 ± 21.34 |
| 50 | 173.71 ± 25.04 | 120.58 ± 32.31 |
| 10 | 172.91 ± 15.75 | 235.11 ± 32.11 |
| relative average | 158.82 ± 25.10 | 242.84 ± 57.30 |
| value | | |

For the determination of the mRNA amount of the heavy antibody chain of the three cell lines with TaqMan hydrolysis probes at first the combination of primers and probe useful in this aspect of the invention had to be determined. The combinations of primers #62, #65, #66, #68, #67, #62, #63 and the TaqMan probes #167 and #168 were tested. The probes contained at the 5′ end the dye IRD700. The PCR products obtained with the different primer-probe-combinations as listed above show (e.g. FIG. 9) that the combinations primers #66 and #68 with probe #168 as well as the combination primers #67 and #68 with probe #168 resulted in PCR products with a high specific product yield and low by-product formation. For increase in the fluorescence intensity the fluorescence dye of probe #168 was changed to Cy5. This new probe was denoted as probe #173. Thus, the primer-probe-combinations #66, #68, and #168 or #173 as well as the primer-probe-combination #67, #68, and #168 or #173 itself are specific aspects of the current invention as well as the use of these primer-probe-combinations in the method according to the invention. In one embodiment is the primer-probe-combination #66, #68, and #173. This combination is preferred as it shows a better PCR efficiency, i.e. a steeper increase of the amplification curve.

The determination of the mRNA amount of the heavy antibody chain of the three cell lines was independently performed four times each with three different mRNA amounts of 250 ng, 50 ng, and 10 ng. The result of one representative experiment obtained with the primer combination #66/#68 and the probe #173 are listed based on the mRNA amount of the single transfected cell line, which was set to 100% relative amount in Table 6. It can be seen, that the cell line 4F5 has approximately 88% more mRNA encoding immunoglobulin heavy chain than the single transfected cell line, and that the cell line 20F2 has approximately 126% more mRNA encoding the immunoglobulin light chain.

TABLE 6
Exemplary results with primer-probe-combination #66/#68/#173.

| amount of mRNA | cell line 4F5 | |
|---|---|---|
| in the sample | % relative to cell line | cell line 20F2 |
| [ng] | 8C8 ± σ | % relative to cell line 8C8 ± σ |
| 250 | 187.47 ± 12.01 | 222.94 ± 19.57 |
| 50 | 190.97 ± 3.74 | 218.86 ± 11.20 |
| 10 | 185.75 ± 6.97 | 234.84 ± 9.06 |
| relative average | 188.06 ± 2.66 | 225.55 ± 8.30 |
| value | | |

The above performed determination method is specific as only a single product is obtained as confirmed by agarose gel electrophoresis.

In order to normalize the results obtained in order to eliminate intraday and interlab variations a correlation to a housekeeping gene can be used. It has been found that the gene encoding the enzyme glyceraldehyde-3-phosphate dehydrogenase (GAPDH) can be used for this purpose. Thus, one aspect of the current invention is the primer-probe-combination #169/#170 and #171 and the use of said combination in a TaqMan probe PCR format for the determination of GAPDH mRNA.

In a multiplex PCR reaction a simultaneous amplification and detection of an mRNA encoding an immunoglobulin heavy chain, an mRNA encoding an immunoglobulin light chain, and an mRNA encoding GAPDH was performed. For the single determination the primer-probe-combinations #132/#133/#166 (light chain, FAM dye), #66/#68/#173 (heavy chain, Cy5 dye), and #169/#170/#171 (GAPDH, Yakima Yellow dye) were used. The combination for the GAPDH gene was not useful in a multiplex PCR reaction. But it has been found that the primer-probe-combination #148/#149/#174 is useful in a multiplex PCR determination of GAPDH mRNA. Thus, one aspect of the current invention is the primer-probe-combination #148/#149 and #174 and the use thereof in a multiplex PCR reaction.

After the multiplex PCR employing the primer-probe-combinations #132/#133/#166 (for light chain amplification and detection, FAM dye), #66/#68/#173 (for heavy chain amplification and detection, Cy5 dye), and #148/#149/#174 (for GAPDH amplification and detection, Yakima Yellow dye) the PCR products were separated on a 2% agarose gel. The detected bands correlated to the expected fragments of 101 by (light chain), 197 by (GAPDH), 244 by (heavy chain) (see FIG. 10).

The efficiency of the real-time PCR reactions was determined based on a dilution series (200 ng, 100 ng, 50 ng, 25 ng, 12.5 ng, 6.25 ng, 3.125 ng) determined as quadruplicates and is given in Table 7.

TABLE 7
Efficiency.

| exper- | Light chain | | | GAPDH | | | Heavy chain | | |
|---|---|---|---|---|---|---|---|---|---|
| iment | 8C8 | 4F5 | 20F2 | 8C8 | 4F5 | 20F2 | 8C8 | 4F5 | 20F2 |
| 1 | 1.905 | 1.884 | 1.951 | 1.94 | 1.983 | 2.069 | 1.949 | 1.997 | 1.992 |
| 2 | 1.971 | 1.936 | 1.936 | 2.064 | 2.067 | 2.085 | 2.043 | 2.027 | 2.037 |
| 3 | 1.924 | 1.945 | 1.936 | 1.989 | 2.097 | 2.041 | 1.963 | 1.905 | 1.991 |
| ø | 1.933 | 1.92 | 1.94 | 2.00 | 2.05 | 2.07 | 1.99 | 1.98 | 2.01 |
| σ | 0.034 | 0.033 | 0.009 | 0.062 | 0.059 | 0.022 | 0.051 | 0.064 | 0.026 |

Thus, an efficiency of 2 for the calculation can be used.

In the multiplex PCR the following amounts for the mRNA encoding the immunoglobulin light chain and the immunoglobulin heavy chain in cell lines 4F5 and 20F2 compared to the cell line 8C8, which is set to 100%, were found.

TABLE 8
Exemplary multiplex PCR results.

| | Light chain | | | | Heavy chain | | | |
|---|---|---|---|---|---|---|---|---|
| ex- | 4F5 | | 20F2 | | 4F5 | | 20F2 | |
| peri- | % of | | % of | | % of | | % of | |
| ment | 8C8 | Dev. | 8C8 | Dev. | 8C8 | Dev. | 8C8 | Dev. |
| 1 | 172.61 | 4.96 | 212.04 | 10.96 | 167.9 | 12.82 | 241.17 | 9.24 |
| 2 | 164.19 | 7.59 | 179.56 | 11.96 | 161.07 | 7.64 | 207.46 | 13.75 |
| 3 | 172.62 | 17.64 | 199.23 | 13.27 | 155.34 | 15.98 | 214.21 | 22.38 |
| ø | 169.81 | 4.86 | 196.94 | 16.36 | 161.44 | 6.29 | 220.95 | 17.84 |

It has now been found that the specific production rate (SPR) of a cell correlates well with the amount of mRNA encoding the produced heterologous polypeptide.

This was found for simplex PCR reactions (Table 9) as well as for multiplex PCR reactions (table 10).

TABLE 9
Exemplary simplex PCR reaction results.

| | SPR | | LC | | HC | |
|---|---|---|---|---|---|---|
| | % Rel- | | % Rel- | | % Rel- | |
| cell | ative | Factor 1 | ative | Factor 2 | ative | Factor 3 |
| 8C8 | 100 | 1 | 100 | 1 | 100 | 1 |
| 4F5 | 185 | 1.85 | 171.51 | 1.71 | 188.06 | 1.88 |
| 20F2 | 166 | 1.66 | 211.4 | 2.11 | 225.55 | 2.26 |

TABLE 10
Exemplary multiplex PCR reaction results.

| | SPR | | Light chain | | Heavy chain | |
|---|---|---|---|---|---|---|
| | % Rel- | | % Rel- | | % Rel- | |
| cell | ative | Factor 1 | ative | Factor 2 | ative | Factor 3 |
| 8C8 | 100 | 1 | 100 | 1 | 100 | 1 |
| 4F5 | 185 | 1.85 | 169.81 | 1.7 | 161.44 | 1.61 |
| 20F2 | 166 | 1.66 | 196.94 | 1.97 | 220.95 | 2.21 |

It has now been found that a factor can be calculated based on the amount of mRNA determined via PCR of a cell with unknown SPR and a cell with known SPR of a heterologous polypeptide which allows for the calculation of the unknown SPR.

TABLE 11
Factor determination.

| | |
|---|---|
| SPR | Factor 1/0.5 * (Factor 2 + 3) |

| cell | % Relative | Simplex PCR | Multiplex PCR |
|---|---|---|---|
| 8C8 | 100 | — | — |
| 4F5 | 185 | 1.0 | 1.1 |
| 20F2 | 166 | 0.8 | 0.8 |

Thus, one aspect of the current invention is a method for determining the productivity of a cell expressing a heterologous polypeptide comprising the steps of
determining the amount of mRNA encoding the heterologous polypeptide in a cell of known productivity,
determining the amount of mRNA encoding the heterologous polypeptide in a cell of unknown productivity,
calculating the ratio of the determined amount of mRNA encoding the heterologous polypeptide of the cell of unknown productivity to the cell of known productivity,
multiplying the productivity of said cell of known productivity with said calculated ratio and thereby determining the productivity of a cell expressing a heterologous polypeptide.

In one embodiment the heterologous polypeptide is an immunoglobulin or an immunoglobulin fragment or an immunoglobulin conjugate. In one embodiment the heterologous immunoglobulin is a multimeric heterologous immunoglobulin. In another embodiment the amount of mRNA encoding the heterologous polypeptide is the sum of the amounts of mRNA encoding all subunits of said heterologous polypeptide divided by the number of subunits. In one embodiment the productivity is the specific production rate in pg/cell/day. In one embodiment the amount of mRNA encoding the heterologous immunoglobulin is the average of the amount of mRNA encoding the light chain of the heterologous immunoglobulin and the amount of mRNA encoding the heavy chain of the heterologous immunoglobulin. In one embodiment the determining of the amount of mRNA is via a polymerase chain reaction (PCR). In one embodiment the PCR is a multiplex PCR. In another embodiment the PCR is a reverse transcription PCR (RT-PCR). In one embodiment the calculated ratio is multiplied by a factor of 0.925.

For example, the specific production rate of a parent cell is 100 pg/cell/day. Via multiplex PCR of the mRNA of a cell of unknown productivity the amount of mRNA encoding the immunoglobulin light chain was determined to be 169% and the amount of mRNA encoding the immunoglobulin heavy chain was determined to be 161% of the amount of mRNA of the parent cell. The average of said mRNA amounts is 165% or 1.65 times the amount of mRNA of the parent cell. Thus, the SPR of the parent cell of 100 pg/cell/day is multiplied by 1.65, thereby obtaining a SPR of 165 pg/cell/day. The SPR of the unknown cell was determined to be 165 pg/cell/day.

The term “about” as used within this application denotes a deviation of +/−10% of the indicated value. Thus, the term “about 1.65” denotes the range of from 1.49 to 1.82.

Depending on the amino acid sequence of the constant region of their heavy chains, antibodies are divided in the classes: IgA, IgD, IgE, IgG, and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ. respectively. The light chain constant regions which can be found in all five antibody classes are called κ (kappa) and λ (lambda).

Due to the different gene copy numbers encoding the heterologous immunoglobulin integrated into the genome the amount of mRNA transcribed from these genes is also different. Thus, a further aspect of the current invention is a method for the determination of the amount of mRNA or DNA with relative quantitation for mRNA or absolute quantitation for DNA comprising
a) providing a sample,
b) performing a polymerase chain reaction with the primers of SEQ ID NO: 23 and 24 and the probe of SEQ ID NO: 33, and/or
c) performing a polymerase chain reaction with the primers of SEQ ID NO: 19 and 21 and the probe of SEQ ID NO: 40, and
d) quantitating with an efficiency of 2.0.

It has furthermore been found that the specific productivity of the different cell lines correlates well with the mRNA amount. It has also been found that the mRNA encoding the heavy chain of the immunoglobulin accounts for 30% of the immunoglobulin encoding mRNA and that the mRNA encoding the light chain of the immunoglobulin accounts for 70% of the immunoglobulin encoding mRNA.

A further aspect of the invention is a method for the selection of an immunoglobulin producing cell comprising
a) providing a cell,
b) isolating the RNA of said cell,
c) performing with the isolated RNA a polymerase chain reaction with the primers of SEQ ID NO: 23 and 24 and the probe of SEQ ID NO: 33,
d) performing with the isolated RNA a polymerase chain reaction with the primers of SEQ ID NO: 19 and 21 and the probe of SEQ ID NO: 40,
e) selecting a cell as an immunoglobulin producing cell if in step c) and d) a polymerase chain reaction product is obtained.

In one embodiment the provided cell has been transfected with a nucleic acid encoding an immunoglobulin. In another embodiment the provided cell is a cell not endogenously producing an immunoglobulin. In one embodiment the cell is a plurality of cells.

Another aspect of the invention is a method for the production of an immunoglobulin comprising
a) providing a plurality of cells,
b) isolating the RNA of each of said cells,
c) performing with the isolated RNA a polymerase chain reaction with the primers of SEQ ID NO: 23 and 24 and the probe of SEQ ID NO: 33,
d) performing with the isolated RNA a polymerase chain reaction with the primers of SEQ ID NO: 19 and 21 and the probe of SEQ ID NO: 40,
e) selecting a cell based on the amount of polymerase chain reaction product formed in step c) and d),
f) cultivating the selected cell,
g) recovering the immunoglobulin from the cell or the culture medium and thereby producing an immunoglobulin.

In one embodiment the cell is selected which has the highest amount of polymerase chain reaction product in step d).

A further aspect of the current invention is a method for the simultaneous determination of IgG1 and IgG4 heavy and light chains in a high throughput manner.

In one embodiment of the current invention is the heterologous polypeptide an anti-Abeta antibody.

In one embodiment of the before presented methods according to the invention the polymerase chain reaction is a TaqMan hydrolysis probe format. In another embodiment said light chain primers are labeled with the dye FAM and the heavy chain primers are labeled with the dye Cy5. In one embodiment the primers of SEQ ID NO: 23 and 24 are for the immunoglobulin light chain and the primers of SEQ ID NO: 19 and 20 are for the immunoglobulin heavy chain. In one embodiment steps c) and d) in addition comprises measuring the amplification of the nucleic acid in real time to determine the amplified amount of the nucleic acid.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Location and direction of primers and probes in the light chain constant region (human IgG kappa chain; SEQ ID NO: 44).

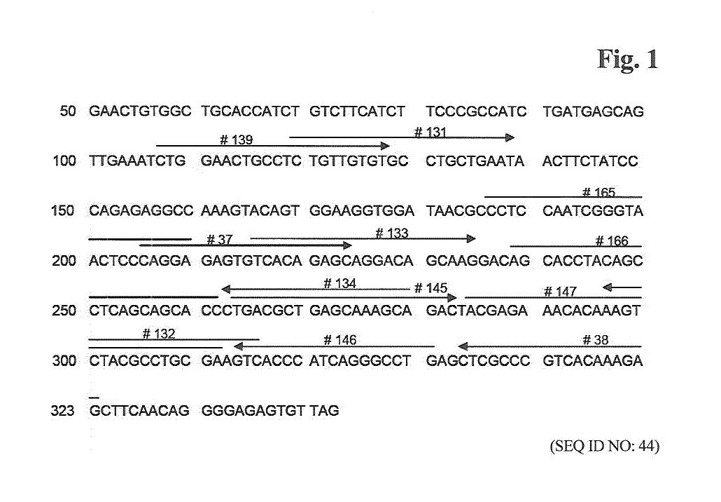

FIG. 2 Location and direction of primers and probes in the heavy chain constant region 1 (human IgG heavy chain CH1; SEQ ID NO: 45).

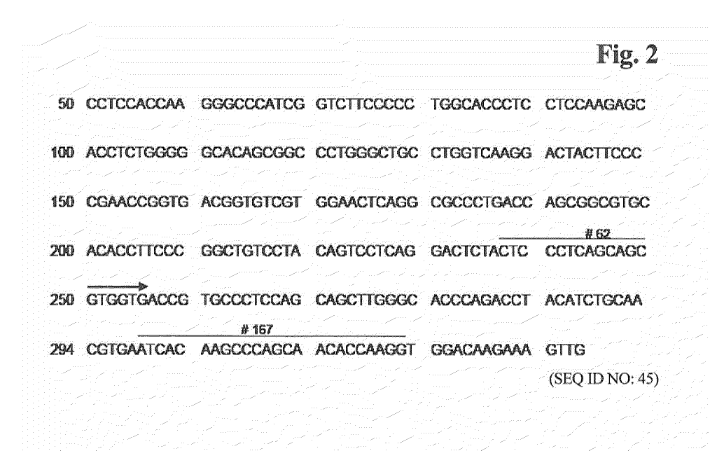

FIG. 3 Location and direction of primers and probes in the heavy chain constant region 2 (human IgG heavy chain CH2; SEQ ID NO: 46).

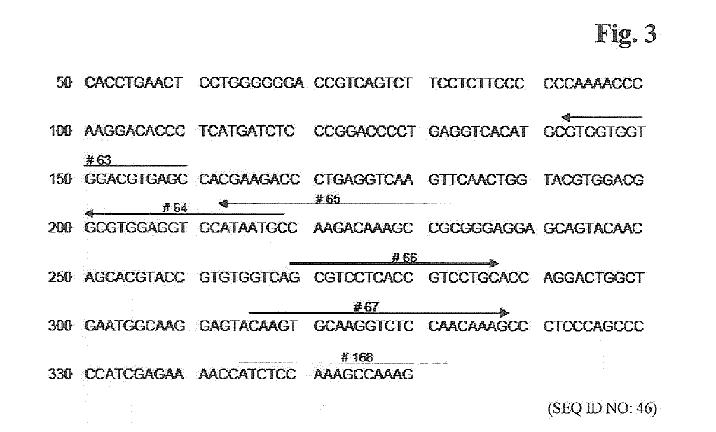

FIG. 4 Location and direction of primers and probes in the heavy chain constant region 3 (human IgG heavy chain CH3; SEQ ID NO: 47).

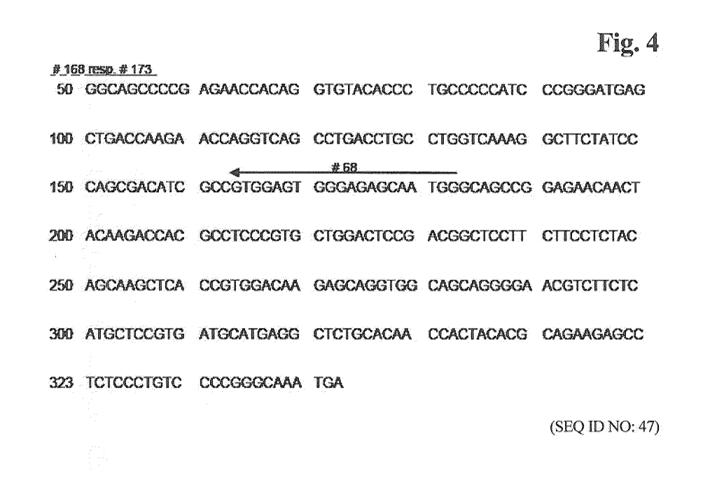

FIG. 5 Agarose gel separation of light chain PCR reaction with the primer combination #131 and #132 and SYBR® GREEN I.

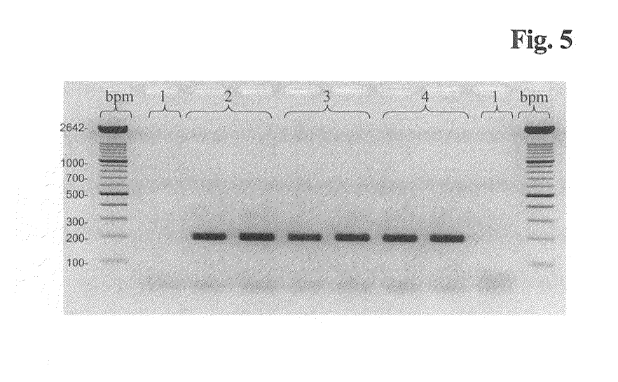

FIG. 6 Agarose gel separation of an 8 μl sample of a 45 cycle PCR reaction; samples: MW: base-pair marker; 1: 139/134-165; 2: 139/134-166; 3: 139/132-165; 4: 139/132-166; 5: 139/146-165; 6: 139/146-166; 7: 139/38-147; 8: 139/38-165; 9: 139/38-166; 10: 139/146-147; 11: 131/38-166; 12: 131/38-147; 13: 37/134-166; 14: 37/132-166; 15: 37/146-166; 16: 37/146-147; 17: 145/146-147; 18: 145/38-147; 19: 131/134-165; 20: 131/134-166; 21: 131/132-165; 22: 131/132-166; 23: 131/146-166; 24: 131/146-165; 25: 131/146-147; 26: 131/38-165; 27: 37/38-166; 28: 133/134-166; 29: 133/132-166; 30: 133/146-166; 31: 133/146-147; 32: 133/38-166.

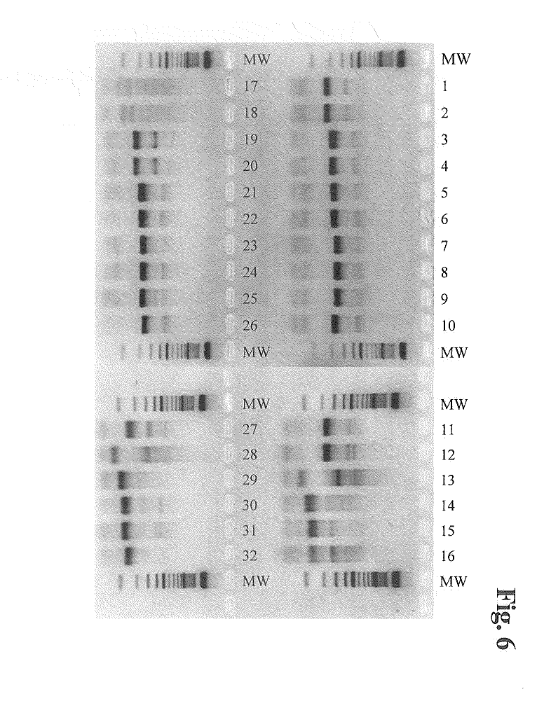

FIG. 7 Amplification curves of PCR reactions with the primer-probe-combinations #133, #132, and #166, or #133/#38, and #160, respectively.

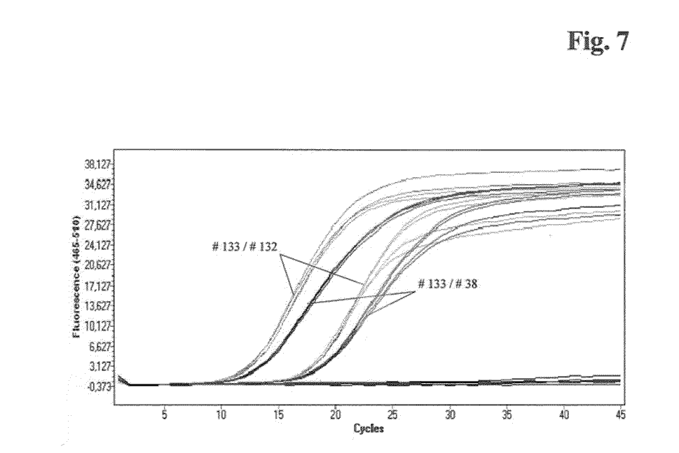

FIG. 8 Agarose gel separation of heavy chain PCR reaction with the primers #62 and #65 and the dye SYBR® Green I; bpm =base pare standard marker; 1: empty reference; 2: 8C8; 3: 4F5; 4: 20F2.

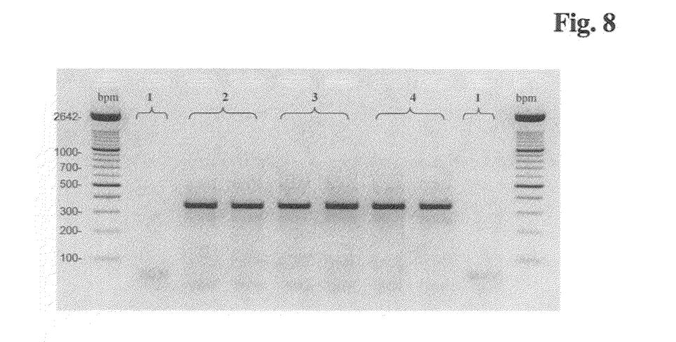

FIG. 9 Agarose gel separation of an 8 μl sample of a 45 cycle PCR reaction; samples: MW: base-pair marker; 1: empty reference; 2: 62/65-167; 3: 66/68-168; 4: 67/68-168.

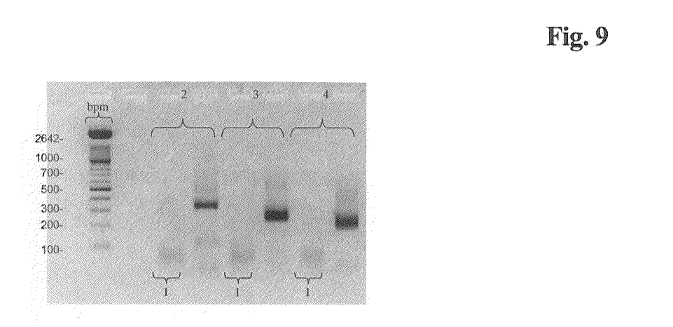

FIG. 10 Agarose gel of the PCR products of a multiplex PCR employing the primer-probe-combinations #132/#133/#166 (for light chain amplification and detection, FAM dye), #66/#68/#173 (for heavy chain amplification and detection, Cy5 dye), and #148/#149/#174 (for GAPDH amplification and detection, Yakima Yellow dye). The detected bands correlated to the expected fragments of 101 by (light chain), 197 by (GAPDH), and 244 by (heavy chain).

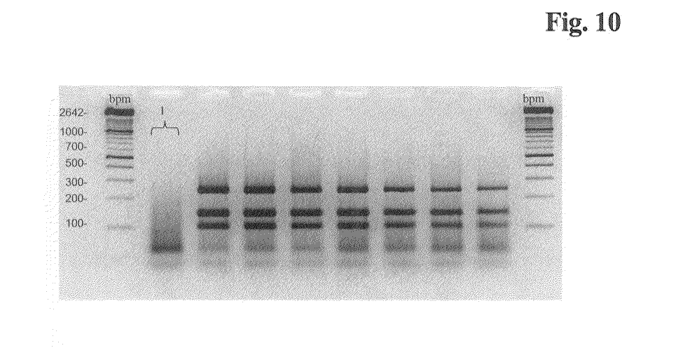

### EXAMPLES

Materials & Methods

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered according to EU numbering (Edelman, G. M., et al., Proc. Natl. Acad. Sci. USA 63 (1969) 78-85; Kabat, E. A., et al., Sequences of Proteins of Immunological Interest, 5th ed., Public Health Service, National Institutes of Health, Bethesda, Md. (1991)).

Recombinant DNA Techniques:

Standard methods were used to manipulate DNA as described in Sambrook, J., et al., Molecular cloning: A laboratory manual; Cold Spring Harbor Laboratory Press, Cold Spring Harbor, N.Y., 1989. The molecular biological reagents were used according to the manufacturer's instructions.

Gene Synthesis:

Desired gene segments were prepared from oligonucleotides made by chemical synthesis. The 100-600 by long gene segments, which are flanked by singular restriction endonuclease cleavage sites, were assembled by annealing and ligation of oligonucleotides including PCR amplification and subsequently cloned into the pCR2.1-TOPO-TA cloning vector (Invitrogen Corp., USA) via A-overhangs or pPCR-Script Amp SK(+) cloning vector (Stratagene Corp., USA). The DNA sequence of the subcloned gene fragments were confirmed by DNA sequencing.

DNA Oligonucleotide Synthesis:

Unlabeled primers and probes, which were labeled with fluorescent dyes and quenchers, were generated by chemical synthesis.

Protein Determination:

Protein concentration was determined by determining the optical density (OD) at 280 nm, using the molar extinction coefficient calculated on the basis of the amino acid sequence.

DNA and RNA Determination:

DNA and RNA concentration was determined by measuring the optical density at 260 nm assuming that an optical density of 1 corresponds to 50 μg/ml double stranded DNA or 40 μg/ml RNA.

Cell Number Determination:

The cell number was determined in a CASY® TT model. Prior to cell number determination the cells were individualized by treatment with trypsin at 37° C. for 10 minutes. Trypsination was terminated by the addition of fetal calf serum (FCS).

Immunoglobulin Titer Determination:

Immunoglobulin titers were determined either by anti-human Fc ELISA or by Protein A chromatography using the autologous purified antibody as a reference.

SDS-PAGE

LDS sample buffer, fourfold concentrate (4×): 4 g glycerol, 0.682 g TRIS-Base, 0.666 g TRIS-hydrochloride, 0.8 g LDS (lithium dodecyl sulfate), 0.006 g EDTA (ethylene diamin tetra acid), 0.75 ml of a 1% by weight (w/w) solution of Serva Blue G250 in water, 0.75 ml of a 1% by weight (w/w) solution of phenol red, add water to make a total volume of 10 ml.

The culture broth containing the secreted immunoglobulin was centrifuged to remove cells and cell debris. An aliquot of the clarified supernatant was admixed with ¼ volumes (v/v) of 4×LDS sample buffer and 1/10 volume (v/v) of 0.5 M 1,4-dithiotreitol (DTT). Then the samples were incubated for 10 min. at 70° C. and protein separated by SDS-PAGE. The NuPAGE® Pre-Cast gel system (Invitrogen Corp., USA) was used according to the manufacturer's instruction. In particular, 10% NuPAGE® Novex® Bis-TRIS Pre-Cast gels (pH 6.4) and a NuPAGE® MOPS running buffer was used.

Western Blot

Transfer buffer: 39 mM glycine, 48 mM TRIS-hydrochloride, 0.04% (w/w) SDS, and 20% (v/v) methanol.

After SDS-PAGE the separated immunoglobulin chains were transferred electrophoretically to a nitrocellulose filter membrane (pore size: 0.45 μm) according to the “Semidry-Blotting-Method” of Burnette (Burnette, W. N., Anal. Biochem. 112 (1981) 195-203).

RNA-Isolation

RNA has been isolated with the RNeasy® mini-Kit from Qiagen (Hilden, Germany) according to the manufacturer's manual. DNA contamination was eliminated by the addition of DNAse. The RNA was isolated from 1×10⁷ cells sampled at the third day of cultivation.

DNA-Isolation

Genomic DNA was isolated with the Blood & Cell Culture DNA Midi Kit from Qiagen (Hilden, Germany) according to the manufacturer's manual from 1×10⁷ cells at the fourth day of cultivation.

Real Time PCR or Real Time RT-PCR

For the real-time PCR or real-time RT-PCR the dyes SYBR® Green I and TaqMan-probes have been used. The reaction mixtures were after preparation and prior to amplification placed on ice in the dark. The determination and analysis was performed with the LightCycler® 2.0-System and LightCycler® software 4.1 or with the LightCycler® II 480-System and LightCycler® software 1.5 (all Roche Diagnostics GmbH, Mannheim, Germany).

### Example 1

Expression Vector for Expressing an Anti-Aβ Antibody

An example antibody with which the methods according to the invention can be exemplified is an antibody against the amyloid β-A4 peptide (anti-Aβ antibody). Such an antibody and the corresponding nucleic acid sequences are, for example, reported in WO 2003/070760 or US 2005/0169925 or in SEQ ID NO: 1 to 12.

Three anti-Aβ antibody expressing Chinese hamster ovary (CHO) cell lines were generated by three successive complete transfections and selection campaigns as reported in WO 2009/046978.

A genomic human κ-light chain constant region gene segment (C-kappa, C_{L}) was added to the light chain variable region of the anti-Aβ antibody, while a human γ1-heavy chain constant region gene segment (C_{H1}-Hinge-C_{H2}-C_{H3}) was added to the heavy chain variable region of the anti-Aβ antibody. The complete κ-light and γ1-heavy chain antibody genes were then joined with a human cytomegalovirus (HCMV) promoter at the 5′-end and a human immunoglobulin polyadenylation signal sequence at the 3′-end.

For expression and production of the anti-Aβ antibody the light and heavy chain expression cassettes were placed on a single expression vector (heavy chain upstream of light chain in clockwise orientation). Three identical expression vectors were generated differing only in the selectable marker gene included, in particular, in the gene conferring resistance to the selection agent neomycin, hygromycin, or puromycin.

The preadapted parent host cells were propagated in suspension in synthetic, animal component-free ProCHO4-complete medium under standard humidified conditions (95%, 37° C., and 5% CO₂). On regular intervals depending on the cell density the cells were splitted into fresh medium. The cells were harvested by centrifugation in the exponential growth phase, washed once in sterile phosphate buffered saline (PBS) and resuspended in sterile PBS.

Prior to transfection the anti-Aβ antibody expressing plasmids were linearized within the β-lactamase gene (E. coli ampicillin resistance marker gene) using the restriction endonuclease enzyme PvuI or AviII. The cleaved DNA was precipitated with ethanol, dried under vacuum, and dissolved in sterile PBS.

In general, for transfection, the CHO cells were electroporated with 20-50 μg linearized plasmid DNA per approximately 10⁷ cells in PBS at room temperature. The electroporations were performed with a Gene Pulser XCell electroporation device (Bio-Rad Laboratories) in a 2 mm gap cuvette, using a square wave protocol with a single 180 V pulse. After transfection, the cells were plated out in ProCHO4-complete medium in 96-well culture plates. After 24 h of growth a solution containing one or more selection agents were added (ProCHO4-complete selection medium; G418: 400 μg/ml; hygromycin: 600 μg/ml; puromycin: 8 μg/ml). Once a week the ProCHO4-complete selection medium was replaced. The antibody concentration of the anti-Aβ antibody was analyzed with an ELISA assay specific for human IgG1 in the culture supernatants.

For selection of anti-Aβ antibody producing cell lines the productivity was tested in ProCHO4-complete selection medium after propagation in 6-well culture plates, T-flasks and/or Erlenmeyer shake flasks using an anti-human IgG1 ELISA and/or analytic Protein A HPLC.

For the first transfection and selection step a plasmid containing a gene conferring resistance to the selection agent neomycin has been used. The plasmid has been transfected with electroporation into parent cell line adapted to growth in ProCHO4-complete medium. The transfected cells were cultivated in ProCHO4-complete medium supplemented with up to 700 μg/ml G418 in 96 well plates. The antibody concentration in the culture supernatants was evaluated by an anti-human IgG1 ELISA. Approximately 1000 clones have been tested and the selected of them were further cultivated in 24-well plates, 6-well plates and subsequently in shaker flasks. The growth and productivity of approximately 20 clones was assessed in static and suspension cultures by anti-human IgG1 ELISA and/or analytic protein A HPLC. The best clone (best clone does not denote the most productive clone it denotes the clone with the best properties for the further steps) was subcloned by limited dilution in ProCHO4-conditioned medium supplemented with 700 μg/ml G418. The selected clone was named 8C8.

For the second transfection and selection step a plasmid containing a gene conferring resistance to the selection agent hygromycin has been used. The plasmid has been transfected with electroporation into cell line cultivated in ProCHO4-complete medium supplemented with 700 μg/ml G418. The transfected cells were expanded for about two to three weeks in ProCHO4-conditioned medium supplemented with 200 μg/ml G418 and 300 μg/ml hygromycin (ProCHO4-double selection medium). Single antibody secreting cells were identified and deposited on the basis of their fluorescence intensity after staining with a Protein A Alexa Fluor conjugate by FACS analysis. The deposited cells were cultivated in ProCHO4-double selection medium in 96 well plates. The antibody concentration in the culture supernatants was evaluated by an anti-human IgG1 ELISA. Approximately 500 clones have been tested and the selected of them were further cultivated in 24-well plates, 6-well plates and subsequently in shaker flasks. The growth and productivity of approximately 14 clones was assessed in static and suspension cultures by anti-human IgG1 ELISA and/or analytic Protein A HPLC. The selected clone was named 4F5.

For the third transfection and selection step a plasmid containing a gene conferring resistance to the selection agent puromycin has been used. The plasmid has been transfected with electroporation into cell line cultivated in ProCHO4-double selection medium. The transfected cells were expanded for about two to three weeks in ProCHO4-triple selection medium (ProCHO4-conditioned medium supplemented with 200 μg/ml G418 and 300 μg/ml hygromycin and 4 μg/ml puromycin). Single antibody secreting cells were identified and deposited on the basis of their fluorescence intensity after staining with a Protein A Alexa Fluor conjugate by FACS analysis. The deposited cells were cultivated in ProCHO4-triple selection medium in 96 well plates. The antibody concentration in the culture supernatants was evaluated by an anti-human IgG1 ELISA. Approximately 500 clones have been tested and the selected of them were further cultivated in 24-well plates, 6-well plates and subsequently in shaker flasks. The growth and productivity of approximately 10 clones was assessed in static and suspension cultures by anti-human IgG1 ELISA and/or analytic protein A HPLC. The selected clone was named 20F2.

Clone Characteristics:

As can be seen from the following table the doubling time and cell density after three days of cultivation were comparable when the basic cell line CHO-K1 (wild-type) and the selected clones are compared.

TABLE 12
Clone characteristics.

| | Doubling | Starting cell | Cell density at | Viability at |
|---|---|---|---|---|
| | time | density | day 3 | day 3 |
| Clone | [h] | [10⁶ cells/ml] | [10⁶ cells/ml] | [%] |
| CHO-K1 | 22-23 | 3 | 18-20 | 97-98 |
| (wild-type) | | | | |
| 8C8 | 26-28 | 3 | 12-15 | 96-98 |
| 4F5 | 22-24 | 3 | 24-27 | 96-97 |
| 20F2 | 24-26 | 2 | 23-26 | 97-98 |

### Example 2

Real-Time RT-PCR with SYBR® Green I

For the RT-PCR with SYBR® Green I the LightCycler® 2.0 system was employed (Roche Diagnostics GmbH, Mannheim, Germany). From the RNA of cell lines 8C8, 4F5 and 20F2 each a dilution series with decreasing RNA concentration was prepared and analyzed. The RNA amount in all samples was supplemented with wild-type-RNA in a way that the total RNA amount, i.e. the sum of wild-type-RNA and sample-RNA, was the same in all samples.

After sample preparation 5 μl of the sample was mixed with 15 μl of a RT-PCR-SG solution. The RT-PCR-SG solution comprises:
5 μl PCR grade water
1.3 μl 50 nM Mn(OAc)₂
7.5 μl SYBR® Green I Pre-Mix
0.6 μl forward primer (10 pmol/μl)
0.6 μl reverse primer (10 pmol/μl).

From each sample three different RNA amounts were analyzed (250 ng, 50 ng, and 10 ng). The PCR conditions were as shown in Table 13.

TABLE 13
PCR conditions.

| | | | | | Ramp | deter- |
|---|---|---|---|---|---|---|
| | | Cycle | T | t | Rate | mina- |
| Program | Phase | number | [° C.] | [min:s] | [° C./s] | tion |
| Reverse | | 1 | 61 | 20:00 | 20 | — |
| Transcription | | | | | | |
| Denaturation | | 1 | 95 | 02:00 | 20 | — |
| Real-Time | Denaturation | 45 | 95 | 00:10 | 20 | — |
| PCR | Annealing | | vari- | 00:20 | 20 | — |
| | | | able | | | |
| | Elongation | | 72 | 00:20 | 2 | — |
| | Detection | | 82 | 00:00 | 20 | single |
| Melting | Denaturation | 1 | 95 | 00:05 | 20 | — |
| curve | Annealing | | 60 | 00:15 | 20 | — |
| | Melting | | 91 | 00:00 | 0.1 | contin- |
| | | | | | | uous |
| cooling | | 1 | 37 | 00:01 | 2.2 | — |

The fluorescence was determined at 530 nm.

Analogously the LightCycler® II 480 system was employed in the RT-PCR. The PCR conditions were as shown in Table 14.

TABLE 14
PCR conditions.

| | | | | | Ramp | deter- |
|---|---|---|---|---|---|---|
| | | cycle | T | t | Rate | mina- |
| Program | phase | number | [° C.] | [min:s] | [° C./s] | tion |
| Reverse | | 1 | 61 | 20:00 | 4.4 | — |
| Transcription | | | | | | |
| Denaturation | | 1 | 95 | 05:00 | 4.4 | — |
| Real-Time | Denaturation | 45 | 95 | 00:10 | 4.4 | — |
| PCR | Annealing | | vari- | 00:20 | 2.2 | — |
| | | | able | | | |
| | Elongation | | 72 | 00:20 | 4.4 | — |
| | Detection | | 82 | 00:00 | 4.4 | single |
| melting | Denaturation | 1 | 95 | 00:05 | 4.4 | — |
| curve | Annealing | | 60 | 01:00 | 2.2 | — |
| | Melting | | 91 | 00:00 | 0.11 | contin- |
| | | | | | | uous |
| cooling | | 1 | 37 | 00:01 | 2.2 | — |

### Example 3

Real-time RT-PCR with TaqMan hydrolysis Probes

For the RT-PCR with TaqMan hydrolysis probes the LightCycler® II 480 system was employed (Roche Diagnostics GmbH, Mannheim, Germany). The PCR samples were prepared by using the LightCycler® 480 RNA Master Hydrolysis Probes Kit (Roche Diagnostics GmbH, Mannheim, Germany).

After sample preparation 5 μl of the sample was mixed with 15 μl of a RT-PCR-HS solution. The RT-PCR-HS solution comprises:
3.8 μl PCR grade water
1.3 μl 3.25 nM Mn(OAc)₂
7.4 μl LightCycler® Pre-Mix
1.0 μl forward primer (10 pmol/μl)
1.0 μl reverse primer (10 pmol/μl)
0.5 μl TaqMan hydrolysis probe (10 pmol/μl).

The PCR conditions were as shown in Table 15.

TABLE 15
PCR conditions.

| | | | | | Ramp | deter- |
|---|---|---|---|---|---|---|
| | | cycle | T | t | Rate | mina- |
| Program | phase | number | [° C.] | [min:s] | [° C./s] | tion |
| Reverse | | 1 | 61 | 20:00 | 4.4 | — |
| Transcription | | | | | | |
| Denaturation | | 1 | 95 | 02:00 | 4.4 | — |
| Real-Time | Denaturation | 45 | 95 | 00:10 | 4.4 | — |
| PCR | Annealing | | 60 | 00:05 | 2.2 | — |
| | Elongation | | 72 | 00:01 | 4.4 | single |
| cooling | | 1 | 37 | 00:01 | 2.2 | — |

### Example 4

Real-time Multiplex RT-PCR with TaqMan Hydrolysis Probes

For the multiplex RT-PCR two or three, respectively, TaqMan hydrolysis probes have been combined. After sample preparation 5 μl of the sample was mixed with 15 μl of a RT-PCR-M_HS solution.

TABLE 16
Components of the RT-PCR-M_HS solution.

volume for

| component | two probes [μl] | three probes [μl |
|---|---|---|
| PCR grade water | 1.3 | 1.3 |
| Mn(OAc)₂, 3.25 mM | 1.3 | 1.3 |
| LightCycler ® Pre-Mix | 7.4 | 7.4 |
| Primer forward 1, 10 pmol/μl | 1 | 0.75 |
| Primer reverse 1, 10 pmol/μl | 1 | 0.75 |
| TaqMan probe 1, 10 pmol/μl | 0.5 | 0.5 |
| Primer forward 2, 10 pmol/μl | 1 | 0.75 |
| Primer reverse 2, 10 pmol/μl | 1 | 0.75 |
| TaqMan probe 2, 10 pmol/μl | 0.5 | 0.5 |
| Primer forward 3, 10 pmol/μl | — | 0.75 |
| Primer reverse 3, 10 pmol/μl | — | 0.75 |
| TaqMan probe 3, 10 pmol/μl | — | 0.5 |
| total | 15 | 15 |

The results of the multiplex RT-PCR have been corrected with a color compensation program generated for the employed TaqMan probes.

### Example 5

Real-time PCR

For the real-time PCR the LightCycler® II 480 system employing SYBR® Green I and TaqMan probes have been used. Each sample was determined in the sample-DNA dilutions 50 ng, 25 ng, 10 ng, 5 ng, and 2.5 ng as quadruplicate. For the real-time PCR 15 μl of the corresponding PCR solution was placed in the well of a 96-well microtiter plate followed by 5 μl of the sample-DNA. The plate was sealed with a LightCycler® 480 sealing foil (Roche Diagnostics GmbH, Mannheim, Germany) and centrifuged at 1,500×g for 2 minutes. Afterwards the plate was mounted into the LightCycler® 480 system. The determination and analysis of the data was done with the LightCycler® 480 software version 1.5.

The copy number was determined by absolute quantitation with the first transfection plasmid of Example 1 as external standard in linearized form.

SYBR® Green I

For the real-time PCR the LightCycler® FastStart Master^{PLUS} SYBR Green I Kit (Roche Diagnostics GmbH, Mannheim, Germany) was employed. The reaction mixture was composed of:
9 μl PCR grade water
4 μl SYBR® Green I Pre-Mix
1 μl forward primer (10 pmol/μl)
1 μl reverse primer (10 pmol/μl).

The employed PCR conditions were as shown in Table 17.

TABLE 17
PCR conditions.

| | | | | | Ramp | deter- |
|---|---|---|---|---|---|---|
| | | cycle | T | t | Rate | mina |
| Program | phase | number | [° C.] | [min:s] | [° C./s] | tion |
| Denaturation | | 1 | 95 | 10:00 | 4.4 | — |
| Real-Time | Denaturation | 45 | 95 | 00:10 | 4.4 | — |
| PCR | Annealing | | 60 | 00:10 | 2.2 | — |
| | Elongation | | 72 | 00:10 | 4.4 | |
| | Detection | | 86 | 00:01 | 4.4 | single |
| cooling | | 1 | 37 | 00:01 | 2.2 | — |

TaqMan Hydrolysis Probe

For the RT-PCR the LightCycler® 480 Probes Master Kit (Roche Diagnostics GmbH, Mannheim, Germany) was used. The reaction mixture was composed of:
2.5 μl PCR grade water
10 μl LightCycler® Pre-Mix
1 μl forward primer (10 pmol/μl)
1 μl reverse primer (10 pmol/μl)
0.5 μl TaqMan hydrolysis probe (10 pmol/μl).

The employed PCR conditions were as shown in Table 18.

TABLE 18
PCR conditions.

| | | | | | Ramp | Deter- |
|---|---|---|---|---|---|---|
| | | Cycle | T | t | Rate | mina- |
| Program | Phase | number | [° C.] | [min:s] | [° C./s] | tion |
| Denaturation | | 1 | 95 | 10:00 | 4.4 | — |
| Real-Time | Denaturation | 45 | 95 | 00:10 | 4.4 | — |
| PCR | Annealing | | 60 | 00:05 | 2.2 | — |
| | Elongation | | 72 | 00:01 | 4.4 | single |
| cooling | | 1 | 37 | 00:01 | 2.2 | — |

Absolute Quantitation

In the absolute quantitation the amount of a nucleic acid sequence is determined in terms of copy number of said sequence. The standard or reference function was determined by analysis of five solutions with known concentrations of the first plasmid used in example 1. The reference function provided for a linear relationship between the Cp value and the copy number of a nucleic acid and allowed for the determination of an unknown copy number in a sample.

The dilutions of the standard samples contained 2.5×10⁷ to 2.5×10² copies of the plasmid. The calculation of the copy number (Nk) of the linearized plasmid of the standard function was done according to the following equations (1) to (4) (see e.g. Jiang, Z., et al., Biotechnol. Prog. 22 (2006) 313-318):

$$\begin{matrix}\begin{matrix}{M}_{Plasmid} = {bp}_{Plasmid} \times {M}_{bp} \\ = 14 \text{,} 0333 bp \cdot 660 g {mol}^{- 1} \\ = 9 \text{,} 261 \text{,} 780 g {mol}^{- 1}\end{matrix} & ( 1 ) \\ {c}_{Plasmid} = 92.92 ng µ {l}^{- 1} ( after linearization ) & ( 2 ) \\ {N}_{A} = 6.022 \times {10}^{23} {mol}^{- 1} ( Avogardo ' s number ) & ( 3 ) \\ {N}_{K} = \frac{{c}_{Plasmid} \cdot {N}_{A}}{{M}_{Plasmid}} = 6.0416 \times {10}^{9} copies µ {l}^{- 1} & ( 4 )\end{matrix}$$

1. A method for determining the amount of mRNA in a sample comprising
a) providing a sample,
b) performing a polymerase chain reaction with the primers of SEQ ID NO: 23 and 24 and the probe of SEQ ID NO: 33, and/or
c) performing a polymerase chain reaction with the primers of SEQ ID NO: 19 and 21 and the probe of SEQ ID NO: 40, and
d) quantitating with an efficiency of 2.0.

2. A method for determining the productivity of a cell expressing an immunoglobulin comprising
a) providing a cell with unkown productivity and a cell with known productivity,
b) performing a polymerase chain reaction with the primers of SEQ ID NO: 23 and 24 and the probe of SEQ ID NO: 33, and/or performing a polymerase chain reaction with the primers of SEQ ID NO: 19 and 21 and the probe of SEQ ID NO: 40 with the RNA of said cell of known productivity and thereby determining the amount of mRNA encoding said immunoglobulin in a cell of known productivity,
c) performing a polymerase chain reaction with the primers of SEQ ID NO: 23 and 24 and the probe of SEQ ID NO: 33, and/or performing a polymerase chain reaction with the primers of SEQ ID NO: 19 and 21 and the probe of SEQ ID NO: 40 with the RNA of said cell of unknown productivity and thereby determining the amount of mRNA encoding said immunoglobulin in a cell of unknown productivity,
d) calculating the ratio of the determined amount of mRNA encoding said immunoglobulin of said cell of unknown productivity to said cell of known productivity,
e) multiplying the productivity of said cell of known productivity with said calculated ratio and thereby determining the productivity of a cell expressing an immunoglobulin.

3. A method for selecting an immunoglobulin producing cell comprising
a) providing a cell,
b) isolating the RNA of said cell,
c) performing with the isolated RNA a polymerase chain reaction with the primers of SEQ ID NO: 23 and 24 and the probe of SEQ ID NO: 33,
d) performing with the isolated RNA a polymerase chain reaction with the primers of SEQ ID NO: 19 and 21 and the probe of SEQ ID NO: 40,
e) selecting a cell as an immunoglobulin producing cell those cells in which a polymerase chain reaction product is formed in steps c) and d).

4. A method for selecting an immunoglobulin producing cell comprising
a) providing a plurality of cells,
b) isolating the RNA from each of said cells,
c) performing with each of the isolated RNA individually a polymerase chain reaction with the primers of SEQ ID NO: 23 and 24 and the probe of SEQ ID NO: 33,
d) performing with each of the isolated RNA individually a polymerase chain reaction with the primers of SEQ ID NO: 19 and 21 and the probe of SEQ ID NO: 40,
e) selecting a cell as immunoglobulin producing those cells in which a polymerase chain reaction product is formed in steps c) and d).

5. A method for the production of an immunoglobulin comprising
a) providing a plurality of cells,
b) isolating the RNA of each of said cells,
c) performing with each of the isolated RNA individually a polymerase chain reaction with the primers of SEQ ID NO: 23 and 24 and the probe of SEQ ID NO: 33,
d) performing with each of the isolated RNA individually a polymerase chain reaction with the primers of SEQ ID NO: 19 and 21 and the probe of SEQ ID NO: 40,
e) selecting a cell based on the amount of polymerase chain reaction product formed in step c) and/or d),
f) cultivating the selected cell,
g) recovering the immunoglobulin from the cell or the culture medium and thereby producing an immunoglobulin.

6. The method of claim 4, wherein the cell is selected that has the highest amount of polymerase chain reaction product in step d).

7. The method of claim 23, wherein the provided cell has or the provided cells have been transfected with a nucleic acid encoding an immunoglobulin.

8. The method claim 2, wherein the ratio in step d) is multiplied with a factor of 0.925.

9. The method of claim 5, wherein the polymerase chain reaction is a TaqMan hydrolysis probe format.

10. The method of claim 5, wherein the primers of SEQ ID NO: 23 and 24 are for the immunoglobulin light chain and the primers of SEQ ID NO: 19 and 20 are for the immunoglobulin heavy chain.

11. The method of claim 10, wherein the light chain primers are labeled with the dye FAM and the heavy chain primers are labeled with the dye Cy5.

12. The method of claim 5, wherein the steps of performing a polymerase chain reaction in addition comprise measuring the amplification of the nucleic acid in real time to determine the amplified amount of the nucleic acid.

13. The method of claim 5, wherein the polymerase chain reaction is a reverse transcriptase polymerase chain reaction.

14. A kit comprising
a) a nucleic acid of SEQ ID NO: 23,
b) a nucleic acid of SEQ ID NO: 24, and
c) a nucleic acid of SEQ ID NO: 33.

15. A kit comprising
a) a nucleic acid of SEQ ID NO: 19,
b) a nucleic acid of SEQ ID NO: 21, and
c) a nucleic acid of SEQ ID NO: 40.

16. (canceled)

17. A nucleic acid of SEQ ID NO: 19.

18. A nucleic acid of SEQ ID NO: 21.

19. A nucleic acid of SEQ ID NO: 23.

20. A nucleic acid of SEQ ID NO: 24.

21. A nucleic acid of SEQ ID NO: 33.

22. A nucleic acid of SEQ ID NO: 40.

23. The method of claim 5, wherein the cell is selected that has the highest amount of polymerase chain reaction product in step d).
